SEQUENCE LISTING (10 sequences)

SEQ ID NO: 1 - protein, 5 aa, synthetic construct
GYYFH

SEQ ID NO: 2 - protein, 17 aa, synthetic construct
RIDPEDDSTKYAERFKD

SEQ ID NO: 3 - protein, 17 aa, synthetic construct
WRIYRDSSGRPFYVMDA

SEQ ID NO: 4 - protein, 11 aa, synthetic construct
LASEDIYTYLT

SEQ ID NO: 5 - protein, 7 aa, synthetic construct
GANKLQD

SEQ ID NO: 6 - protein, 9 aa, synthetic construct
LQGSKFPLT

SEQ ID NO: 7 - protein, 126 aa, synthetic construct
QVQLVQSGAEVKKPGASVKVSCKVSGEISTGYYFHWVRQAPGKGLEWMGRIDPEDDSTKYAERFKDRVTMTEDTSTDTAYMELSSLRSEDTAVYYCTTWRIYRDSSGRPFYVMDAWGQGTLVTVSS

SEQ ID NO: 8 - protein, 107 aa, synthetic construct
DIQMTQSPSSVSASVGDRVTITCLASEDIYTYLTWYQQKPGKAPKLLIYGANKLQDGVPSRFSGSGSGTDYTLTISSLQPEDFATYYCLQGSKFPLTFGQGTKLEIK

SEQ ID NO: 9 - protein, 456 aa, synthetic construct
QVQLVQSGAEVKKPGASVKVSCKVSGEISTGYYFHWVRQAPGKGLEWMGRIDPEDDSTKYAERFKDRVTMTEDTSTDTAYMELSSLRSEDTAVYYCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSVSASVGDRVTITCLASEDIYTYLTWYQQKPGKAPKLLIYGANKLQDGVPSRFSGSGSGTDYTLTISSLQPEDFATYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0234237 A1
(43) Pub. Date: Aug. 13, 2026

(54) METHODS OF TREATING ATOPIC DERMATITIS

(71) Applicant: GLAXOSMITHKLINE INTELLECTUAL PROPERTY DEVELOPMENT LIMITED, Stevenage (GB)

(72) Inventors: Nuria Buil-Bruna, Stevenage (GB); Robert A. Scott, Stevenage (GB); Iain J. Uings, Stevenage (GB)

(73) Assignee: GLAXOSMITHKLINE INTELLECTUAL PROPERTY DEVELOPMENT LIMITED, Stevenage (GB)

(21) Appl. No.: 19/156,963

(22) PCT Filed: Feb. 22, 2024
(86) PCT No.: PCT/IB2024/051737

§ 371 Date: Aug. 15, 2025

Related U.S. Application Data
(60) Provisional application No. 63/486,752, filed on Feb. 24, 2023.

(51) Int. Cl. C07K 16/24; A61K 9/00; A61P 17/00

(52) U.S. Cl. CPC C07K 16/244; A61K 9/0019; A61P 17/00

(57) ABSTRACT

The disclosure provides a method of treating atopic dermatitis in a human subject by the administration of certain anti-IL-18 antibodies. The disclosure also provides uses of certain anti-IL-18 antibodies in the treatment of atopic dermatitis in a human subject.

### FIELD

The present disclosure generally relates to methods of treating atopic dermatitis in a human subject by the administration of an anti-interleukin-18 (IL-18) antibody.

### BACKGROUND

Atopic dermatitis (also known as eczema) is a chronic inflammatory disease that causes dry, itchy, and inflamed skin. Itchy skin (also known as pruritus) is one of the essential features of atopic dermatitis and the diagnosis of active atopic dermatitis cannot be made without a history of itching. Because of the high impact on life quality, most patients measure the severity of eczema by the intensity of pruritus and/or the appearance of skin lesions. Conventional treatments for atopic dermatitis include topical therapies, phototherapy, and systemic therapies. Topical therapies such as topical calcineurin inhibitors and topical phosphodiesterase-4 inhibitors are commonly used for treating mild disease but have limited efficacy in moderate or severe disease. While systemic therapies such as corticosteroids and immunosuppressants demonstrate efficacy, adverse effects and poor tolerability limit their long-term use. Accordingly, there continues to be a need for effective therapies to treat atopic dermatitis subjects.

### SUMMARY

In one aspect, the disclosure provides a method of treating atopic dermatitis in a human subject in need thereof, the method comprising: administering a dose of about 1 mg/kg to about 5 mg/kg of an anti-interleukin-18 (IL-18) antibody to the human subject, wherein the anti-IL-18 antibody comprises a heavy chain variable region (VH) comprising a heavy chain complementarity determining region (CDRH) 1, a CDRH2, and a CDRH3 set forth as SEQ ID NOs: 1, 2, and 3, respectively; a light chain variable region (VL) comprising a light chain complementarity determining region (CDRL) 1, a CDRL2, and a CDRL3 set forth as SEQ ID NOs: 4, 5, and 6, respectively. In some embodiments, the VH comprises the amino acid sequence of SEQ ID NO: 7. In some embodiments, the VL comprises the amino acid sequence of SEQ ID NO: 8. In some embodiments, the anti-IL-18 antibody comprises a heavy chain as set forth in SEQ ID NO: 9 and a light chain as set forth in SEQ ID NO: 10.

In one aspect, there is provided a method of treating pruritus in a human subject in need thereof, the method comprising: administering a dose of about 1 mg/kg to about 5 mg/kg of an anti-interleukin-18 (IL-18) antibody to the human subject, wherein the anti-IL-18 antibody comprises a heavy chain variable region (VH) comprising a heavy chain complementarity determining region (CDRH) 1, a CDRH2, and a CDRH3 set forth as SEQ ID NOs: 1, 2, and 3, respectively; a light chain variable region (VL) comprising a light chain complementarity determining region (CDRL) 1, a CDRL2, and a CDRL3 set forth as SEQ ID NOs: 4, 5, and 6, respectively. In some embodiments, the VH comprises the amino acid sequence of SEQ ID NO: 7. In some embodiments, the VL comprises the amino acid sequence of SEQ ID NO: 8. In some embodiments, the anti-IL-18 antibody comprises a heavy chain as set forth in SEQ ID NO: 9 and a light chain as set forth in SEQ ID NO: 10.

In one aspect, there is provided an anti-interleukin-18 (IL-18) antibody for use in the treatment of atopic dermatitis or pruritus in a human subject in need thereof, wherein the anti-IL-18 antibody comprises a heavy chain variable region (VH) comprising a heavy chain complementarity determining region (CDRH) 1, a CDRH2, and a CDRH3 set forth as SEQ ID NOs: 1, 2, and 3, respectively; a light chain variable region (VL) comprising a light chain complementarity determining region (CDRL) 1, a CDRL2, and a CDRL3 set forth as SEQ ID NOs: 4, 5, and 6, respectively, wherein the anti-IL-18 antibody is administered to the human subject with a dose of about 1 mg/kg to about 5 mg/kg. In some embodiments, the VH comprises the amino acid sequence of SEQ ID NO: 7. In some embodiments, the VL comprises the amino acid sequence of SEQ ID NO: 8. In some embodiments, the anti-IL-18 antibody comprises a heavy chain as set forth in SEQ ID NO: 9 and a light chain as set forth in SEQ ID NO: 10.

In one aspect, there is provided use of an anti-IL18 antibody in the manufacture of a medicament for the treatment of atopic dermatitis or pruritus, wherein the anti-IL-18 antibody comprises a heavy chain variable region (VH) comprising a heavy chain complementarity determining region (CDRH) 1, a CDRH2, and a CDRH3 set forth as SEQ ID NOs: 1, 2, and 3, respectively; a light chain variable region (VL) comprising a light chain complementarity determining region (CDRL) 1, a CDRL2, and a CDRL3 set forth as SEQ ID NOs: 4, 5, and 6, respectively, wherein the anti-IL-18 antibody is administered to the human subject with a dose of about 1 mg/kg to about 5 mg/kg. In some embodiments, the VH comprises the amino acid sequence of SEQ ID NO: 7. In some embodiments, the VL comprises the amino acid sequence of SEQ ID NO: 8. In some embodiments, the anti-IL-18 antibody comprises a heavy chain as set forth in SEQ ID NO: 9 and a light chain as set forth in SEQ ID NO: 10.

It is understood that any and all embodiments of the present disclosure may be taken in combination with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### BRIEF DESCRIPTION OF THE FIGURES

FIG. 1 is a graph depicting the change from baseline in peak pruritis numerical rating scale (PP-NRS) score for subjects receiving placebo (solid line, closed circle) or IL-18 antibody 2 mg/kg (dashed line, closed triangle). Posterior median and 95% Credible Interval (CrI) of change from baseline in PP-NRS score for biologic naïve subjects are shown; PRO: patient-reported outcomes.

FIG. 2 is a graph depicting the change from baseline in dermatology life quality index (DLQI) score for subjects receiving placebo (solid line, closed circle) or IL-18 antibody 2 mg/kg (dashed line, closed triangle). Posterior median and 95% CrI of change from baseline in DLQI score for biologic naïve subjects are shown.

### DETAILED DESCRIPTION

### Definitions

As used herein, the term “about” means within +10% of the stated value.

The term “antigen binding protein” as used herein refers to antibodies and other protein constructs, such as domains, which are capable of binding to the antigen.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin.

Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342:877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 1 below represents one definition using each numbering convention for each CDR or binding unit. It should be noted that some of the CDR definitions may vary depending on the individual publication used.

TABLE 1

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

The term, “percent identity” or “% identity” or “sequence identity” between a query nucleic acid sequence/amino acid sequence and a subject nucleic acid sequence/amino acid sequence is the “Identities” value, expressed as a percentage, that is calculated using a suitable algorithm (e.g., BLASTN, FASTA, Needleman-Wunsch, Smith-Waterman, LALIGN, or GenePAST/KERR) or software (e.g., DNASTAR Lasergene, GenomeQuest, EMBOSS needle or EMBOSS infoalign), over the entire length of the query sequence after a pair-wise global sequence alignment has been performed using a suitable algorithm (e.g., Needleman-Wunsch or GenePAST/KERR) or software (e.g., DNASTAR Lasergene or GenePAST/KERR). Importantly, a query nucleic acid sequence/amino acid sequence may be described by a nucleic acid sequence/amino acid sequence disclosed herein, in particular in one or more of the claims. The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. In the case of amino acid sequences, such alterations include at least one amino acid residue deletion, substitution (including conservative and non-conservative substitutions), or insertion, wherein said alterations may occur at the amino- or carboxy-terminal positions of the query sequence or anywhere between those terminal positions, interspersed either individually among the amino acid residues in the query sequence or in one or more contiguous groups within the query sequence. For antibody sequences, the % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g., the framework sequence, so that the CDR sequences are fixed and intact.

Ranges provided herein include all values within a particular range described and values about an endpoint for a particular range. The figures and tables of the disclosure also describe ranges, and discrete values, which may constitute an element of any of the methods and uses disclosed herein. Also, where ranges and/or subranges of values are provided, the ranges and/or subranges can include the endpoints of the ranges and/or subranges.

“Treating” as used herein refers to alleviating one or more symptoms or effects associated with a disorder and/or slowing the progression of the disorder.

“Biosimilar” as used herein refers to a biopharmaceutical or a biologic product that is highly similar to a reference biologic product (e.g., dupilumab) notwithstanding minor differences in clinically inactive components, and for which there are no clinically meaningful differences between the biologic product and the reference product in terms of the safety, purity, and potency of the product (Section 351 (i) of the Public Health Service Act (42 U.S.C. 262 (i))).

Therefore, the term “biosimilar” refers to a biologic product that is highly similar to the reference product (e.g., dupilumab) approved by a regulatory agency (e.g., the Federal Drug Administration (FDA) or the European Medicines Agency (EMA)) based on data from (a) analytical studies demonstrating that the biologic product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biologic product and the reference product in terms of the safety, purity, and potency of the product). In certain embodiments, the biosimilar product is an interchangeable product as determined by a regulatory agency (e.g., the FDA).

### Treatment of Atopic Dermatitis

Described herein are methods of treating atopic dermatitis or pruritus in a human subject that benefits from administration of anti-IL-18 antibodies. Also described are uses of anti-IL-18 antibodies in the manufacture of a medicament for use in treating atopic dermatitis in a human subject. Further described are uses of anti-IL-18 antibodies in the treatment of atopic dermatitis or pruritus in a human subject.

In one aspect, the disclosure provides methods of treating atopic dermatitis in a human subject, the method comprising: administering a dose of about 1 mg/kg to about 5 mg/kg of an anti-interleukin-18 (IL-18) antibody to a human subject in need thereof.

In one aspect, the disclosure provides the use of a dose of about 1 mg/kg to about 5 mg/kg of an anti-interleukin-18 (IL-18) antibody in the manufacture of a medicament for use in treating atopic dermatitis in a human subject in need thereof.

In one aspect, the disclosure provides the use of a dose of about 1 mg/kg to about 5 mg/kg of an anti-interleukin-18 (IL-18) antibody in the treatment of atopic dermatitis in a human subject in need thereof.

In some embodiments, the anti-IL-18 antibody comprises a heavy chain and a light chain. In some embodiments, the anti-IL-18 antibody is a monoclonal antibody. In some embodiments, the anti-IL-18 antibody is an IgG12 monoclonal antibody.

In some embodiments, the anti-IL-18 antibody comprises a heavy chain and a light chain and the heavy chain comprises a CDRHI as set forth in SEQ ID NO: 1, a CDRH2 as set forth in SEQ ID NO: 2, and a CDRH3 as set forth in SEQ ID NO: 3. In some embodiments, the anti-IL-18 antibody comprises a heavy chain and a light chain and the light chain comprises a CDRL1 as set forth in SEQ ID NO: 4, a CDRL2 as set forth in SEQ ID NO: 5, and a CDRL3 as set forth in SEQ ID NO: 6. In some embodiments, the anti-IL-18 antibody comprises a heavy chain and a light chain and the heavy chain comprises a CDRH1 as set forth in SEQ ID NO: 1, a CDRH2 as set forth in SEQ ID NO: 2, and a CDRH3 as set forth in SEQ ID NO: 3, and the light chain comprises a CDRLI as set forth in SEQ ID NO: 4, a CDRL2 as set forth in SEQ ID NO: 5, and a CDRL3 as set forth in SEQ ID NO: 6.

In some embodiments, the anti-IL-18 antibody comprises a heavy chain and a light chain and the heavy chain comprises a heavy chain variable region comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 7. In some embodiments, the anti-IL-18 antibody comprises a heavy chain and a light chain and the light chain comprises a light chain variable region comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 8. In some embodiments, the anti-IL-18 antibody comprises a heavy chain and a light chain and the heavy chain comprises a heavy chain variable region as set forth in SEQ ID NO: 7 and the light chain comprises a light chain variable region as set forth in SEQ ID NO: 8.

In some embodiments, the anti-IL-18 antibody comprises a heavy chain and a light chain and the heavy chain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9. In some embodiments, the anti-IL-18 antibody comprises a heavy chain and a light chain and the light chain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10. In some embodiments, the anti-IL-18 antibody comprises a heavy chain as set forth in SEQ ID NO: 9 and a light chain as set forth in SEQ ID NO: 10.

In some embodiments, the dose is defined as an amount of the antibody relative to the body weight of the human subject (i.e., mg/kg). The methods comprise administering a dose of about 1 mg/kg to about 5 mg/kg of an anti-IL-18 antibody to a human subject. The uses comprise a dose of about 1 mg/kg to about 5 mg/kg of an anti-IL-18 antibody formulated for administration to a human subject. In some embodiments, the dose is about 1 mg/kg to about 4 mg/kg, about 1 mg/kg to about 3 mg/kg, about 1 mg/kg to about 2 mg/kg, about 2 mg/kg to about 3 mg/kg, about 3 mg/kg to about 4 mg/kg, about 4 mg/kg to about 5 mg/kg, about 1 mg/kg, about 2 mg/kg, about 3 mg/kg, about 4 mg/kg, and/or about 5 mg/kg of an anti-IL-18 antibody. In some embodiments, the dose is about 1 mg/kg to about 3 mg/kg of an anti-IL-18 antibody. In some embodiments, the dose is about 2 mg/kg of an anti-IL-18 antibody.

In some embodiments, the methods comprise administering a dose of about 50 mg to about 500 mg of an anti-IL-18 antibody to a human subject. In some embodiments, the uses comprise a dose of about 50 mg to about 500 mg of an anti-IL-18 antibody formulated for administration to a human subject.

In some embodiments, the dose is about 100 mg to about 400 mg, about 100 mg to about 300 mg, about 50 mg to about 100 mg, about 100 mg to about 200 mg, about 200 mg to about 300 mg, about 300 mg to about 400 mg, about 400 mg to about 500 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, and/or about 500 mg of an anti-IL-18 antibody. In some embodiments, the dose is about 150 mg of an anti-IL-18 antibody.

In some embodiments, the anti-IL-18 antibody is administered by parenteral administration, i.e., subcutaneously (s.c. or SC), intrathecally, intraperitoneally, intramuscularly (i.m. or IM) or intravenously (i.v. or IV), including by intravenous infusion. In some embodiments, the anti-IL-18 antibody is administered intravenously. In some embodiments, the anti-IL-18 antibody is administered through an intravenous infusion.

In some embodiments, the anti-IL-18 antibody is formulated for parenteral administration, i.e., subcutaneous (s.c. or SC), intrathecal, intraperitoneal, intramuscular (i.m. or IM) or intravenous (i.v. or IV) administration, including by intravenous infusion. In some embodiments, the anti-IL-18 antibody is formulated for intravenous administration. In some embodiments, the anti-IL-18 antibody is formulated for intravenous infusion.

In some embodiments, the human subject has not previously received any therapy for the treatment of atopic dermatitis (e.g., the human subject is biologic or drug naïve). Biologic therapies include, but are not limited to, treatments with proteinaceous molecules including antibodies, nucleic acids, cell-based therapies, and vaccines. Biologic naïve subject includes any human subject who has not previously received any biologic therapies for the treatment of atopic dermatitis. In some embodiments, the human subject has not previously received any agent selected from the group consisting of: dupilumab, lebrikizumab, tralokinumab, abrocitinib, and upadacitinib, or any biosimilars or analogs thereof. In some embodiments, the human subject has not previously received dupilumab or any biosimilars thereof for treatment of atopic dermatitis.

In some embodiments, the anti-IL-18 antibody is administered to the human subject who has been previously treated with at least one other therapy for the treatment of atopic dermatitis. In some embodiments, the at least one other therapy comprises at least one agent selected from the group consisting of: dupilumab, lebrikizumab, tralokinumab, abrocitinib, and upadacitinib, or any biosimilars or analogs thereof. In some embodiments, the anti-IL-18 antibody is administered to a human subject who has been previously treated with at least one other agent selected from the group consisting of: dupilumab, abrocitinib, and tralokinumab, or any biosimilars or analogs thereof. In some embodiments, the anti-IL-18 antibody is administered to a human subject who has been previously treated with dupilumab or any biosimilars thereof. In some embodiments, the human subject previously received dupilumab. In some embodiments, the human subject had an inadequate response to treatment with dupilumab (i.e., the human subject is an inadequate dupilumab responder). Inadequate response to treatment with dupilumab includes, but is not limited to, inadequate response following at least 16 weeks of treatment with dupilumab according to the Investigator's judgement, as well as intolerance to dupilumab owing to adverse events.

In some embodiments, the human subject who has been previously treated with at least one other therapy for the treatment of atopic dermatitis has received the other therapy at least one day prior to the treatment with the anti-IL-18 antibody according to any one of the embodiments as disclosed herein. In some embodiments, the human subject who has been previously treated with at least one other therapy for the treatment of atopic dermatitis has received the other therapy at least 7 days, 28 days, 56 days, or 84 days prior to the treatment with the anti-IL-18 antibody according to any one of the embodiments as disclosed herein. In some embodiments, the at least one other therapy comprises at least one agent selected from the group consisting of: dupilumab, lebrikizumab, tralokinumab, abrocitinib, and upadacitinib, or any biosimilars or analogs thereof. In some embodiments, the anti-IL-18 antibody is administered to a human subject who has been previously treated with at least one other agent selected from the group consisting of: dupilumab, abrocitinib, and tralokinumab, or any biosimilars or analogs thereof. In some embodiments, the anti-IL-18 antibody is administered to a human subject who has been previously treated with dupilumab or any biosimilars thereof.

In some embodiments, the atopic dermatitis is moderate to severe atopic dermatitis. In some embodiments, the atopic dermatitis is moderate atopic dermatitis. In some embodiments, the atopic dermatitis is severe atopic dermatitis. Disease severity is determined according to conventional methods including the Investigator Global Assessment (IGA) score and the Eczema Area and Severity Index (EASI) score.

In some embodiments, the human subject has a reduced eczema area and severity index (EASI) score from baseline at week 12 after administration of the anti-IL-18 antibody. In some embodiments, the EASI score is reduced by at least 25%, at least 30%, at least 40%, at least 50%, at least 60%, at least 70%, at least 75%, at least 80%, at least 85%, or at least 90% from baseline at week 12 after administration of the anti-IL-18 antibody.

In some embodiments, the human subject has a reduced Investigator Global Assessment (IGA) score from baseline at week 12 after administration of the anti-IL-18 antibody. In some embodiments, the IGA score is reduced by at least 25%, at least 30%, at least 40%, at least 50%, at least 60%, at least 70%, at least 75%, at least 80%, at least 85%, or at least 90% from baseline at week 12 after administration of the anti-IL-18 antibody.

In some embodiments, the human subject has a reduced peak pruritis numerical rating scale (PP-NRS) score from baseline at week 12 after administration of the anti-IL-18 antibody. In some embodiments, the PP-NRS score is reduced by at least 25%, at least 30%, at least 40%, at least 50%, at least 60%, at least 70%, at least 75%, at least 80%, at least 85%, or at least 90% from baseline at week 12 after administration of the anti-IL-18 antibody.

In some embodiments, the human subject has a reduced Dermatology Life Quality Index (DLQI) score from baseline at week 12 after administration of the anti-IL-18 antibody. In some embodiments, the DLQI score is reduced by at least 25%, at least 30%, at least 40%, at least 50%, at least 60%, at least 70%, at least 75%, at least 80%, at least 85%, or at least 90% from baseline at week 12 after administration of the anti-IL-18 antibody.

### Anti-IL-18 Antibodies

The present disclosure is directed to methods of treating atopic dermatitis in a human subject by administering an anti-IL-18 antibody and to uses of an anti-IL-18 antibody in the treatment of atopic dermatitis in a human subject. The anti-IL-18 antibodies of the disclosure include biosimilars thereof.

The term “anti-interleukin-18” or “anti-IL-18” as it refers to antibodies of the disclosure means that such antibodies are capable of neutralizing the biological activity of human IL-18. It does not exclude, however, that such antibodies may also neutralize the biological activity of non-human primate (e.g., rhesus and/or cynomolgus) IL-18.

In some embodiments, the anti-IL-18 antibody comprises the heavy chain CDR1, heavy chain CDR2, heavy chain CDR3, light chain CDR1, light chain CDR2, and light chain CDR3 sequences of SEQ ID NO: 1, SEQ ID NO: 2, SEQ ID NO: 3, SEQ ID NO: 4, SEQ ID NO: 5, and SEQ ID NO: 6, respectively. In some embodiments, the anti-IL-18 antibody comprises the heavy and light variable chain sequences of SEQ ID NO: 7 and SEQ ID NO: 8, respectively. In some embodiments, the anti-IL-18 antibody comprises the heavy and light chain sequences of SEQ ID NO: 9 and SEQ ID NO: 10, respectively.

| SEQ ID | | |
|---|---|---|
| NO | Name | Sequence |
| 1 | CDRH1 | GYYFH |
| 2 | CDRH2 | RIDPEDDSTKYAERFKD |
| 3 | CDRH3 | WRIYRDSSGRPFYVMDA |
| 4 | CDRL1 | LASEDIYTYLT |
| 5 | CDRL2 | GANKLQD |
| 6 | CDRL3 | LQGSKFPLT |
| 7 | VH | QVQLVQSGAEVKKPGASVKVSCKVSG |
| | | EISTGYYFHWVRQAPGKGLEWMGRI |
| | | DPEDDSTKYAERFKDRVTMTEDTST |
| | | DTAYMELSSLRSEDTAVYYCTTWRI |
| | | YRDSSGRPFYVMDAWGQGTLVTVSS |
| 8 | VL | DIQMTQSPSSVSASVGDRVTITCLA |
| | | SEDIYTYLTWYQQKPGKAPKLLIYG |
| | | ANKLQDGVPSRFSGSGSGTDYTLTI |
| | | SSLQPEDFATYYCLQGSKFPLTFG |
| | | QGTKLEIK |
| 9 | HC | QVQLVQSGAEVKK |
| | | PGASVKVSCKVSGEISTGYYFHWVR |
| | | QAPGKGLEWMGRIDPEDDSTKYAER |
| | | FKDRVTMTEDTSTDTAYMELSSLRS |
| | | EDTAVYYCTTWRIYRDSSGRPFYVM |
| | | DAWGQGTLVTVSSASTKGPSVFPLA |
| | | PSSKSTSGGTAALGCLVKDYFPEPV |
| | | TVSWNSGALTSGVHTFPAVLQSSGL |
| | | YSLSSVVTVPSSSLGTQTYICNVNH |
| | | KPSNTKVDKKVEPKSCDKTHTCPPC |
| | | PAPELLGGPSVFLFPPKPKDTLMIS |
| | | RTPEVTCVVVDVSHEDPEVKFNWYV |
| | | DGVEVHNAKTKPREEQYNSTYRVVS |
| | | VLTVLHQDWLNGKEYKCKVSNKALP |
| | | APIEKTISKAKGQPREPQVYTLPPS |
| | | RDELTKNQVSLTCLVKGFYPSDIAV |
| | | EWESNGQPENNYKTTPPVLDSDGSF |
| | | FLYSKLTVDKSRWQQGNVFSCSVMH |
| | | EALHNHYTQKSLSLSPGK |
| 10 | LC | DIQMTQSPSSVSASVGDRVTITCLA |
| | | SEDIYTYLTWYQQKPGKAPKLLIYG |
| | | ANKLQDGVPSRFSGSGSGTDYTLTI |
| | | SSLQPEDFATYYCLQGSKFPLTFGQ |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |

### Doses of Anti-IL-18 Antibodies

In some embodiments, the dose is in an amount of the antibody (mg) relative to body weight (kg) of the human subject (e.g., mg/kg). In some embodiments, the dose of the anti-IL-18 antibody is within a range of about 1 mg/kg to about 5 mg/kg of human body weight. The dose can be about 1 mg/kg to about 5 mg/kg of total body weight (e.g., about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, or a range defined by any two of the foregoing values). In some embodiments, the dose is about 1 mg/kg to about 4 mg/kg, about 1 mg/kg to about 3 mg/kg, about 1 mg/kg to about 2 mg/kg, about 2 mg/kg to about 3 mg/kg, about 3 mg/kg to about 4 mg/kg, about 4 mg/kg to about 5 mg/kg, about 1 mg/kg, about 2 mg/kg, about 3 mg/kg, about 4 mg/kg, and/or about 5 mg/kg. In some embodiments, the dose is about 1 mg/kg to about 3 mg/kg. In some embodiments, the dose is about 2 mg/kg.

In some embodiments, the dose is in a fixed amount. In some embodiments, the dose of the anti-IL-18 antibody is within a range of about 50 to about 500 mg (e.g., about 50 mg, about 60 mg, about 70 mg, about 75 mg, about 80 mg, about 90 mg, about 100 mg, about 150 mg, about 200 mg, about 250 mg, about 300 mg, about 350 mg, about 400 mg, about 450 mg, about 500 mg, or a range defined by any two of the foregoing values). In some embodiments, the dose of the anti-IL-18 antibody is about 100 mg to about 400 mg, about 100 mg to about 300 mg, about 50 mg to about 100 mg, about 100 mg to about 200 mg, about 200 mg to about 300 mg, about 300 mg to about 400 mg, about 400 mg to about 500 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, and/or about 500 mg. In some embodiments, the dose of the anti-IL-18 antibody is about 150 mg.

In some embodiments, there is provided a method of treating atopic dermatitis in a human subject in need thereof, the method comprising: administering a dose of about 1 mg/kg to about 5 mg/kg of an anti-interleukin-18 (IL-18) antibody to the human subject, wherein the anti-IL-18 antibody comprises a heavy chain variable region (VH) comprising a heavy chain complementarity determining region (CDRH) 1, a CDRH2, and a CDRH3 set forth as SEQ ID NOs: 1, 2, and 3, respectively; a light chain variable region (VL) comprising a light chain complementarity determining region (CDRL) 1, a CDRL2, and a CDRL3 set forth as SEQ ID NOs: 4, 5, and 6, respectively. In some embodiments, the VH comprises the amino acid sequence of SEQ ID NO: 7. In some embodiments, the VL comprises the amino acid sequence of SEQ ID NO: 8. In some embodiments, the anti-IL-18 antibody comprises a heavy chain as set forth in SEQ ID NO: 9 and a light chain as set forth in SEQ ID NO: 10.

In some embodiments, there is provided a method of treating pruritus in a human subject in need thereof, the method comprising: administering a dose of about 1 mg/kg to about 5 mg/kg of an anti-interleukin-18 (IL-18) antibody to the human subject, wherein the anti-IL-18 antibody comprises a heavy chain variable region (VH) comprising a heavy chain complementarity determining region (CDRH) 1, a CDRH2, and a CDRH3 set forth as SEQ ID NOs: 1, 2, and 3, respectively; a light chain variable region (VL) comprising a light chain complementarity determining region (CDRL) 1, a CDRL2, and a CDRL3 set forth as SEQ ID NOs: 4, 5, and 6, respectively. In some embodiments, the VH comprises the amino acid sequence of SEQ ID NO: 7. In some embodiments, the VL comprises the amino acid sequence of SEQ ID NO: 8. In some embodiments, the anti-IL-18 antibody comprises a heavy chain as set forth in SEQ ID NO: 9 and a light chain as set forth in SEQ ID NO: 10.

In some embodiments according to any methods disclosed herein, the dose is about 1 mg/kg to about 3 mg/kg. In some embodiments, the dose is about 1.5 mg/kg to about 2.5 mg/kg. In some embodiments, the dose is about 1 mg/kg, about 2 mg/kg, or about 3 mg/kg. In some embodiments, the dose is about 2 mg/kg.

In some embodiments, the anti-IL-18 antibody is administered intravenously. In some embodiments, the anti-IL-18 antibody is administered through an intravenous infusion.

In some embodiments according to any methods disclosed herein, the human subject maintains a reduced eczema area and severity index (EASI) score compared to the initial baseline control for at least about 4 weeks, about 8 weeks, or about 12 weeks after the administration of the anti-IL-18 antibody.

In some embodiments according to any methods disclosed herein, the administration of the anti-IL-18 antibody reduces itchy skin (or pruritus) of the human subject. In some embodiments, the itchy skin (or pruritus) of the human subject is reduced at least 4 weeks after the administration of the anti-IL-18 antibody.

In one aspect, there is provided an anti-interleukin-18 (IL-18) antibody for use in the treatment of atopic dermatitis in a human subject in need thereof. In some embodiments, the anti-IL-18 antibody comprises a heavy chain variable region (VH) comprising a heavy chain complementarity determining region (CDRH) 1, a CDRH2, and a CDRH3 set forth as SEQ ID NOs: 1, 2, and 3, respectively; a light chain variable region (VL) comprising a light chain complementarity determining region (CDRL) 1, a CDRL2, and a CDRL3 set forth as SEQ ID NOs: 4, 5, and 6, respectively, wherein the anti-IL-18 antibody is administered to the human subject with a dose of about 1 mg/kg to about 5 mg/kg.

In some embodiments, there is provided an anti-interleukin-18 (IL-18) antibody for use according to any one of the preceding embodiments, wherein the VH comprises the amino acid sequence of SEQ ID NO: 7. In some embodiments according to any one of the preceding embodiments, wherein the VL comprises the amino acid sequence of SEQ ID NO: 8.

In some embodiments, there is provided an anti-IL-18 antibody for use according to any one of the preceding embodiments, wherein the anti-IL-18 antibody comprises a heavy chain as set forth in SEQ ID NO: 9 and a light chain as set forth in SEQ ID NO: 10.

In some embodiments, there is provided an anti-IL-18 antibody for use according to any one of the preceding embodiments, wherein the dose is about 1 mg/kg to about 3 mg/kg, or wherein the dose is about 1.5 mg/kg to about 2.5 mg/kg, or wherein the dose is about 1 mg/kg, about 2 mg/kg, or about 3 mg/kg, or wherein the dose is about 2 mg/kg.

In some embodiments, there is provided an anti-IL-18 antibody for use according to any one of preceding embodiments, wherein the anti-IL-18 antibody is administered intravenously, or wherein the anti-IL-18 antibody is administered through an intravenous infusion.

In some embodiments, there is provided an anti-IL-18 antibody for use according to any one of preceding embodiments, wherein the anti-IL-18 antibody is administered to a human subject who has not previously received any therapy for the treatment of atopic dermatitis. In some embodiments, the human subject is biologic or drug naïve.

In some embodiments, there is provided an anti-IL-18 antibody for use according to any one of preceding embodiments, wherein the anti-IL-18 antibody is administered to the human subject who has been previously treated with at least one other therapy for the treatment of atopic dermatitis. In some embodiments, the at least one other therapy comprises at least one agent selected from the group consisting of: dupilumab, lebrikizumab, tralokinumab, abrocitinib, and upadacitinib, or any biosimilars or analogs thereof. In some embodiments, the anti-IL-18 antibody is administered to a human subject who has been previously treated with at least one other agent selected from the group consisting of: dupilumab, abrocitinib, and tralokinumab, or any biosimilars or analogs thereof. In some embodiments, the anti-IL-18 antibody is administered to a human subject who has been previously treated with dupilumab or any biosimilars thereof.

In some embodiments, there is provided an anti-IL-18 antibody for use according to any one of preceding embodiments, wherein the atopic dermatitis is moderate to severe atopic dermatitis.

In some embodiments, there is provided an anti-IL-18 antibody for use according to any one of preceding embodiments, wherein the human subject maintains a reduced eczema area and severity index (EASI) score compared to the initial baseline control for at least about 4 weeks, about 8 weeks, or about 12 weeks after the administration of the anti-IL-18 antibody.

In some embodiments, there is provided an anti-IL-18 antibody for use according to any one of preceding embodiments, wherein the administration of the anti-IL-18 antibody reduces pruritus of the human subject. In some embodiments, the pruritus of the human subject is reduced at least 4 weeks after the administration of the anti-IL-18 antibody.

In some embodiments according to any one of the preceding embodiments, the human subject who has been previously treated with at least one other therapy for the treatment of atopic dermatitis has received the other therapy at least one day prior to the treatment with the anti-IL-18 antibody as disclosed herein. In some embodiments, the human subject who has been previously treated with at least one other therapy for the treatment of atopic dermatitis has received the other therapy at least 7 days, 28 days, 56 days, or 84 days prior to the treatment with the anti-IL-18 antibody as disclosed herein. In some embodiments, the at least one other therapy comprises at least one agent selected from the group consisting of: dupilumab, lebrikizumab, tralokinumab, abrocitinib, and upadacitinib, or any biosimilars or analogs thereof. In some embodiments, the anti-IL-18 antibody is administered to a human subject who has been previously treated with at least one other agent selected from the group consisting of: dupilumab, abrocitinib, and tralokinumab, or any biosimilars or analogs thereof. In some embodiments, the anti-IL-18 antibody is administered to a human subject who has been previously treated with dupilumab or any biosimilars thereof.

The following examples are provided to illustrate, but not limit the claimed disclosure.

### EXAMPLES

### Example 1: Administration of Anti-IL-18 Antibody to Subjects Having Moderate to Severe Atopic Dermatitis

An anti-IL-18 antibody (“mAb01”) was given intravenously at a dose of 2 mg/kg on a single occasion to subjects having moderate to severe atopic dermatitis. The clinical effect was monitored for a 12-week treatment period with an additional 12-week follow-up period.

The subjects were either “biologic naïve” (n=30) or had an inadequate response to dupilumab (“Dupi-IR”) defined by investigator judgement after at least 16 weeks of treatment or because of intolerability due to adverse events (n=4). Because of the small size of the study, dynamic borrowing of historical placebo (PBO) data was utilized to bolster the statistical analysis in the biologic naïve group.

Safety: No serious adverse events (SAEs) occurred. In total, 31 adverse events were reported, 16 in subjects on PBO and 15 in subjects on active.

Efficacy: The primary endpoint was the percent change from baseline (PCFB) in the eczema areas and severity index (EASI) in the biologic naïve group at week 12 and the results are shown in Table 2. Crl: Credible Interval.

TABLE 2

| | PCFB in EASI @ week 12 |
|---|---|
| Treatment | posterior median (95% CrI) |
| Placebo | −32.81 (−46.59, −20.34) |
| N = 10 | |
| mAb01 2 mg/kg | −66.11 (−78.65, −55.54) |
| N = 20 | |
| Difference vs PBO | −33.21 (−50.96, −14.84) |

The percentage of subjects who achieved a 50%, 75%, or 90% reduction in the EASI score in the biologic naïve group at week 12 is shown in Table 3.

TABLE 3

| | EASI 50 response | EASI 75 response | EASI 90 response |
|---|---|---|---|
| | rate (%) | rate (%) | rate (%) |
| | Posterior median | Posterior median | Posterior median |
| Treatment | (95% CrI) | (95% CrI) | (95% CrI) |
| Placebo | 29.0 (8.0, 60.1) | 9.4 (0.7, 35.7) | 7.8 (0.5, 33.2) |
| N = 10 | | | |
| mAb01 2 mg/kg | 71.2 (49.3, 87.8) | 34.6 (16.4, 56.7) | 20.3 (6.0, 42.4) |
| N = 20 | | | |
| Difference vs | 41.1 (4.2, 70.5) | 23.8 (−6.3, 49.4) | 11.0 (−14.5, 35.1) |
| PBO | | | |

The percentage of patients who achieved an investigator global assessment (IGA) score of 0/1 (“clear” or “almost clear”) in the biologic naïve group at week 12 is shown in Table 4.

TABLE 4

| | IGA0/1 response |
|---|---|
| | rate (%) |
| | Posterior median |
| Treatment | (95% CrI) |
| Placebo | 10.2 (0.8, 36.9) |
| N = 10 | |
| mAb01 2 mg/kg | 25.0 (9.9, 46.2) |
| N = 20 | |
| Difference vs PBO | 13.8 (−15.5, 38.4) |

The PCFB in the EASI in the Dupi-IR group at week 12 is shown in Table 5.

TABLE 5

| | PCFB in EASI @ week 12 |
|---|---|
| Treatment | mean observed |
| Placebo | −38.868 |
| N = 1 | |
| mAb01 2 mg/kg | −94.213 |
| N = 2 | |

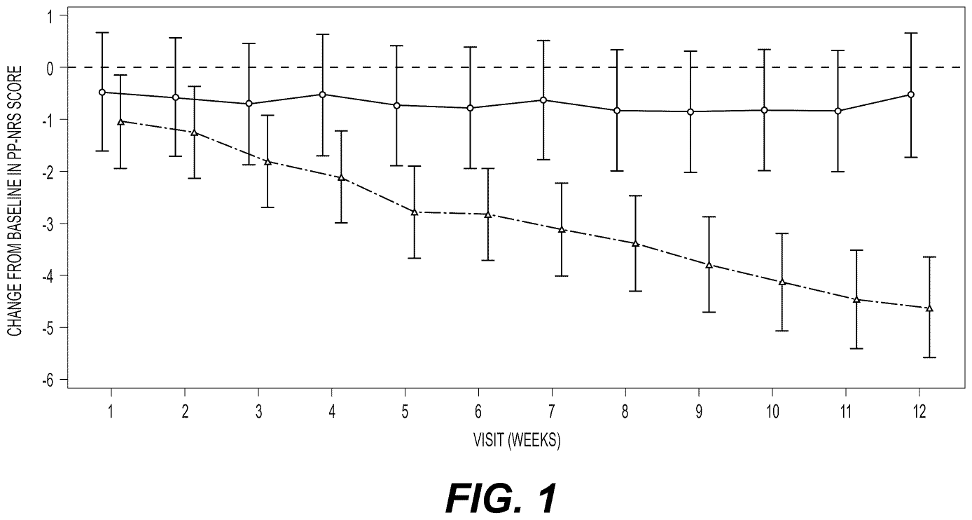

An exploratory endpoint included patient-reported outcomes (PROs) of the peak pruritis numerical rating scale (PP-NRS). PP-NRS is a measurement of itch reported as the absolute change from baseline in the PP-NRS score in the biologic naïve group at week 12. The baseline PP-NRS scores were 7.57±2.001 in the placebo group and 7.79±1.551 in the active group. The results are shown in Table 6. The change from baseline over time is shown in FIG. 1.

TABLE 6

| | PP-NRS |
|---|---|
| | Change from baseline |
| Treatment | Posterior median (95% CrI) |
| Placebo | −0.52 (−1.73, 0.67) |
| N = 10 | |
| mAb01 2 mg/kg | −4.61 (−5.58, −3.65) |
| N = 20 | |
| Difference vs PBO | −4.10 (−5.63, −2.55) |

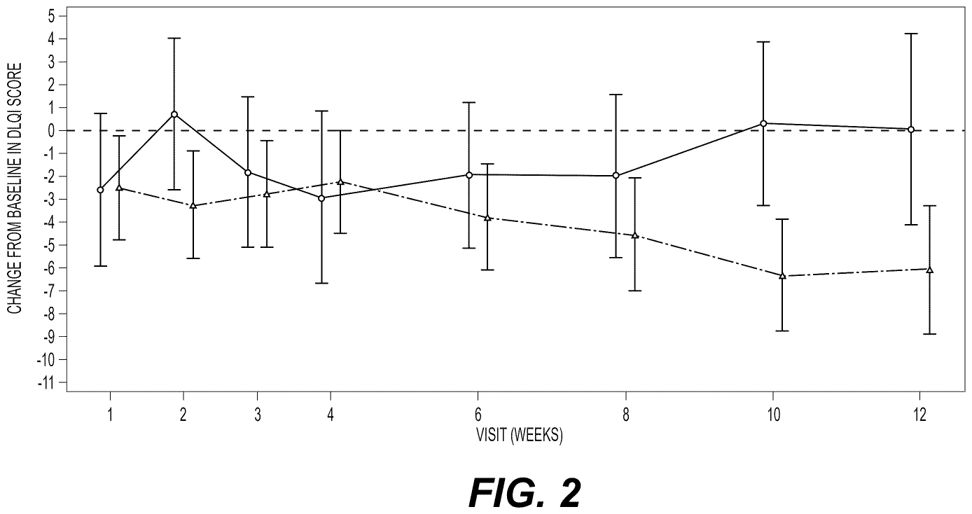

Another exploratory endpoint included PROs of the dermatology life quality index (DLQI). DLQI is reported as the absolute change from baseline in the DLQI score in the biologic naïve group at week 12. The baseline DLQI scores were 13.1±11.98 in the placebo group and 11.1±5.61 in the active group. The results are shown in Table 7. The change from baseline over time is shown in FIG. 2.

TABLE 7

| | DLQI |
|---|---|
| | Change from baseline |
| Treatment | Posterior median (95% CrI) |
| Placebo | 0.05 (−4.08, 4.22) |
| N = 10 | |
| mAb01 2 mg/kg | −6.06 (−8.86, −3.29) |
| N = 20 | |
| Difference vs PBO | −6.13 (−11.41, −0.90) |

The results demonstrated that mAb01 was well-tolerated and effective in biologic naïve subjects across the primary and secondary efficacy endpoint and provide evidence of a beneficial effect in DUPI-IR patients. The results also demonstrated a favourable treatment effect on validated PROs measuring itch (PP-NRS) and quality of life (DLQI).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims.

1. A method of treating atopic dermatitis in a human subject in need thereof, the method comprising: administering a dose of about 1 mg/kg to about 5 mg/kg of an anti-interleukin-18 (IL-18) antibody to the human subject, wherein the anti-IL-18 antibody comprises a heavy chain variable region (VH) comprising a heavy chain complementarity determining region (CDRH) 1, a CDRH2, and a CDRH3 set forth as SEQ ID NOs: 1, 2, and 3, respectively; a light chain variable region (VL) comprising a light chain complementarity determining region (CDRL) 1, a CDRL2, and a CDRL3 set forth as SEQ ID NOs: 4, 5, and 6, respectively.

2. The method of claim 1, wherein the VH comprises the amino acid sequence of SEQ ID NO: 7.

3. The method of claim 1, wherein the VL comprises the amino acid sequence of SEQ ID NO: 8.

4. The method of claim 1, wherein the anti-IL-18 antibody comprises a heavy chain as set forth in SEQ ID NO: 9 and a light chain as set forth in SEQ ID NO: 10.

5. The method of claim 1, wherein the dose is about 1 mg/kg to about 3 mg/kg.

6. The method of claim 1, wherein the dose is about 1.5 mg/kg to about 2.5 mg/kg.

7. The method of claim 1, wherein the dose is about 1 mg/kg, about 2 mg/kg, or about 3 mg/kg.

8. The method of claim 7, wherein the dose is about 2 mg/kg.

9. The method of claim 1, wherein the anti-IL-18 antibody is administered intravenously.

10. (canceled)

11. The method of claim 1, wherein the anti-IL-18 antibody is administered to a human subject who has not previously received any therapy for the treatment of atopic dermatitis.

12. The method of claim 1, wherein the anti-IL-18 antibody is administered to a human subject who has been previously treated with at least one other agent selected from the group consisting of: dupilumab, abrocitinib, and tralokinumab, or any analogs or any biosimilars thereof.

13. The method of claim 12, wherein the at least one other agent is dupilumab or any biosimilars thereof.

14. The method of claim 1, wherein the atopic dermatitis is moderate to severe atopic dermatitis.

15. The method of claim 1, wherein the human subject maintains a reduced eczema area and severity index (EASI) score compared to the initial baseline control for at least about 4 weeks, about 8 weeks, or about 12 weeks after the administration of the anti-IL-18 antibody.

16. The method of claim 1, wherein the administration of the anti-IL-18 antibody reduces pruritus of the human subject.

17. The method of claim 16, wherein the pruritus of the human subject is reduced at least 4 weeks after the administration of the anti-IL-18 antibody.

18.-34. (canceled)

35. A method of treating atopic dermatitis in a human subject in need thereof, the method comprising: administering a dose of about 1 mg/kg to about 5 mg/kg of an anti-interleukin-18 (IL-18) antibody to the human subject, wherein the anti-IL-18 antibody comprises a heavy chain as set forth in SEQ ID NO: 9 and a light chain as set forth in SEQ ID NO: 10, wherein the eczema area and severity index (EASI) score of the human subject from baseline at week 12 after administration of the anti-IL-18 antibody is reduced by at least 40%.

36. The method of claim 35, wherein the human subject has a reduced Investigator Global Assessment (IGA) score from baseline at week 12 after administration of the anti-IL-18 antibody of at least 40%.

37. The method of claim 35, wherein the human subject has been previously treated with dupilumab or any biosimilars thereof.

38. The method of claim 35, wherein the atopic dermatitis is moderate to severe atopic dermatitis.
